(19) United States
(12) Patent Application Publication
(10) Pub. No.: US 2013/0035588 A1
(43) Pub. Date: Feb. 7, 2013

(54) MAGNETIC RESONANCE IMAGING FOR THERAPY PLANNING

(71) Applicants: Steven Michael Shea, Baltimore, MD (US); Erik John Tryggestad, Baltimore, MD (US)

(72) Inventors: Steven Michael Shea, Baltimore, MD (US); Erik John Tryggestad, Baltimore, MD (US)

(73) Assignee: Siemens Corporation, Iselin, NJ (US)

(21) Appl. No.: 13/549,685

(22) Filed: Jul. 16, 2012

Related U.S. Application Data
(60) Provisional application No. 61/514,547, filed on Aug. 3, 2011.

(51) Int. Cl. A61B 5/055

(52) U.S. Cl. 600/413

(57) ABSTRACT

Magnetic resonance imaging (MRI) is used for therapy planning. The motion or position of the treatment region is tracked over time for many cycles using MRI. For temporal resolution, the tracking is done in planes through the tumor at different orientations rather than using three-dimensional scanning. The tracking may be used for calculating a spatial probability density function for the target. Alternatively or additionally, spatiotemporal information derived from the surrogate is compared directly to that from the tracked object to determine the accuracy or robustness of the surrogate-to-target 3D correlation Gating or tracking based on this surrogate may be performed where the comparison indicates that the surrogate is sufficiently reliable (accurate).

### CROSS-REFERENCE TO RELATED APPLICATION

This application claims the benefit of U.S. provisional application entitled “Four Dimensional (4D) Tracking System Using Orthogonal Dynamic 2D MRI,” filed Aug. 3, 2011, and assigned Ser. No. 61/514,547, the entire disclosure of which is hereby incorporated by reference.

### BACKGROUND

The present embodiments relate to dynamic medical imaging systems. Magnetic resonance imaging (MRI) is a medical imaging technique in widespread use for viewing the structure and function of the human body. MRI systems provide soft-tissue contrast, which may be useful for diagnosing soft-tissue disorders, such as tumors.

Anatomic motion due to normal respiration represents a formidable challenge in radiotherapy, both for accurate treatment (dose) planning and for delivery since such motion may lead to a discrepancy between planned and actual target positions. Four dimensional (i.e., three spatial and time) computed tomography (CT) is the emerging gold standard for determining target (tumor) location over time and to derive a 3D (or 4D) dose distribution which avoids healthily tissue. The major drawback of 4D-CT is that it is based on a single-respiratory cycle snapshot (in time) at each axial position and therefore may fail to address normal breathing variability. 4D-CT imparts radiation dose to patients, so repetition of 4D-CT is avoided.

One strategy to account for respiratory motion is either define a generic uncertainty margin surrounding the target for radiotherapy treatments delivered with patients breathing freely. This uncertainty margin is either based on consensus knowledge for large patient populations or, more recently, uses each patient's 4D-CT study.

In another strategy, respiratory gating is provided. Respiration is monitored during imaging and treatment and the pre-treatment scan (e.g., 4D-CT) is used to infer the target location at any given point in the respiratory cycle. One such technique measures respiration with a surrogate (e.g., respiratory belt or an optically monitored external fiducial). The same “gating window” in each cycle is then used for the treatment delivery.

In a related strategy, the patient holds their breath (either voluntarily or under assistance) to effectively arrest breathing motion while the radiotherapy is being administered. This strategy typically relies on reproducibility of target position as a function of lung-air exchange (via spirometry).

In other strategies, the tumor or treatment location is tracked. In one example, markers are implanted into the tumor region, and dual-source cine x-ray imaging is used to track those markers during treatment. However, x-rays impart imaging dose to the patient, and implantation of the markers is invasive. In yet another example, a combined radiation therapy irradiation system and MRI tracks the tumor motion in potentially 4D space. However, 3D MRI may not provide sufficient temporal resolution.

Each of these techniques has other drawbacks due to variance of the anatomic motion from cycle-to-cycle or breath hold to breath hold. Margin increases may be based on the general population. As a result, large margins are non-patient specific and may result in under-irradiation of the tumor or over-irradiation of large areas of healthy tissue. Shifts of tumor position over time due to respiratory drift between the external surrogate and the actual tumor may result in the given strategy being less effective. 4D-CT only measures one or a few respiratory cycles and combines data over respiratory cycles, which may result in severe image artifacts due to inconsistent respiratory motion across the acquisition. Additionally, respiratory motion may often continue to change in the subsequent periods after 4D-CT is completed, thus under-representing the true 4D tumor motion. Longer acquisitions for 4D-CT to capture such data are technically possible, but cannot be done due to radiation dose concerns or restrictions.

### SUMMARY

By way of introduction, the embodiments described below include methods, systems, and computer readable media for therapy planning using magnetic resonance imaging (MRI). The motion or position of the treatment region is tracked over time for many cycles using MRI. For temporal resolution, the tracking is done in planes through the tumor at different orientations rather than using three-dimensional scanning. The tracking may be used for calculating the tumor spatial 3D probability density function. Alternatively or additionally, the tracking is used to compare with the surrogate motion or signal to establish the long-term surrogate-to-tumor correspondence. Gating may be performed where the comparison indicates gating as appropriate for the given patient. Margins may be established based on the tracked object.

In a first aspect, a method is provided for therapy planning using magnetic resonance imaging (MRI). Magnetic resonance (MR) data representing first and second planes at different times is acquired over a plurality of physiological cycles. The first and second planes intersect through an object in a patient and are non-parallel. A processor tracks a position of the object along first and second directions in the first plane from the MR data representing the first plane. The processor tracks the position of the object along third and fourth directions in the second plane from the MR data representing the second plane. A breathing signal is also measured The processor compares a position based on the measured physiological cycle with the position as tracked over time in at least the second direction. An allowance of therapy using the measuring of the physiological cycle is verified based on the comparing. The position from the tracking is incorporated into a probability density function for the therapy.

In a second aspect, a system is provided for therapy planning using image tracking. A respiratory monitor acquires surrogate respiratory data over a plurality of respiratory cycles. A scanner acquires frame data over the plurality of the respiratory cycles. The frame data includes first and second pluralities of frames representing first and second orthogonal planes, respectively, at different times. One or more processors are in communication with the respiratory monitor and the scanner. The one or more processors are configured to determine motion in the first and second planes from the first and second pluralities of the frames, respectively, to calculate differences between the motion determined from the frames and a motion from the surrogate respiratory data over the plurality of the respiratory cycles, and to indicate a feasibility of gating treatment based on the differences.

In a third aspect, a non-transitory computer readable storage medium has stored therein data representing instructions executable by a programmed processor for therapy planning using magnetic resonance imaging (MRI). The storage medium includes instructions for locating position as a function of time of an object represented in MR data for different planes through a patient, calculating probability density functions for different phases of a respiratory cycle as a function of the position over time, and accounting for drift over respiratory cycles including the respiratory cycle as a function of the position over the time.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow diagram of an example embodiment of a method for therapy planning using magnetic resonance imaging (MRI);

FIGS. 2A and 2B illustrate, from different directions, a relative position of two planes to a treatment region;

FIG. 3 shows different images based on acquired MR data;

FIG. 4 is an example graph of position over time along different directions; and

FIG. 5 is a block diagram of an example embodiment of a magnetic resonance imaging (MRI) system configured to implement therapy planning using magnetic resonance imaging (MRI).

### DETAILED DESCRIPTION OF THE PRESENTLY PREFERRED EMBODIMENTS

Two dimensional (2D) magnetic resonance (MR) images of a tumor are repeatedly acquired over time. The images represent two or more non-parallel (e.g., orthogonal) planes through the tumor. A respiratory waveform may also be acquired with a respiratory belt, navigator images, or self-gating techniques. The 2D slice planes may be acquired sequentially over and over again while simultaneously recording external surrogate motion. The tumor is then tracked via 2D template matching or similar techniques to produce four dimensional (4D-3D in space and 1D in time) information on tumor position. This information is used to (1) determine subject specific tumor 3D spatial probability density functions for radiotherapy planning, (2) set uncertainty margins for free-breathing treatments, and/or (3) determine which particular motion management strategy is likely the safest (e.g., which of gating, breath-holding or tracking would perform best for a given situation) from the point of view of target volume minimization.

Since MRI is used, invasive placement of fiducials may be avoided. MRI does not use ionizing radiation. Without increasing radiation dose, imaging may occur over longer time durations. This longer duration may better capture respiratory drift that occurs on the order of minutes and therefore be more representative, temporally, of a typical radiotherapy treatment. The methods and systems may include one or more respiration-correlated averaging procedures to address the variability, or non-reproducibility, present in respiration. Without increasing radiation dose, inter-fraction (i.e., between radiation therapy doses) MRI scans may be performed to assess if respiratory motion has changed and to perform quality and assessment of the current radiation therapy treatment plan.

Using the 4D tracking based on planar MRI, more sophisticated motion-compensation techniques may be employed in therapy planning, and radiotherapy treatment volumes may shrink as a result. The shrinkage may facilitate dose escalation to improve local tumor control as well as reduce radiation toxicity to adjacent normal tissues at risk.

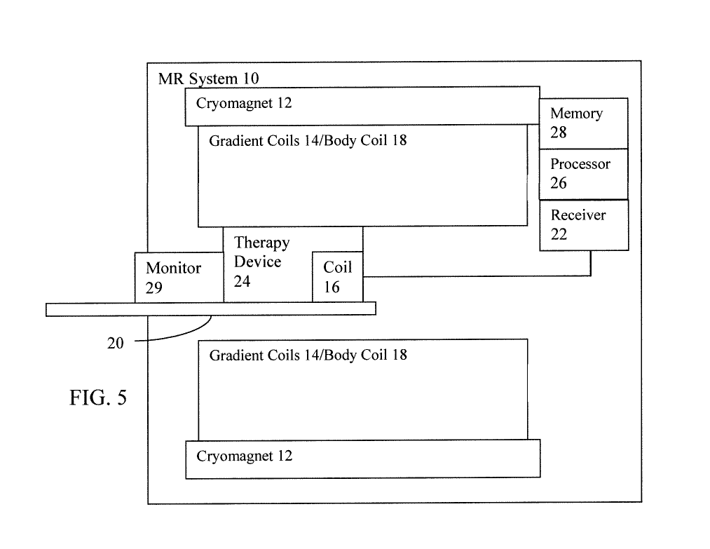

FIG. 1 shows one embodiment of a method for therapy planning using magnetic resonance imaging (MRI). The method is implemented with the system of FIG. 5 or another system. A processor, such as for an imaging system, workstation, or computer, may perform various of the acts, such as acts 62, 64, 66, 68, and 70. Combinations of processors, systems, imaging devices, therapy devices, or other components may be used to implement the acts, such as performing act 60 with an MRI system, performing acts 62-70 with a processor, and performing act 72 with a radiation therapy system.

The acts are performed in the order shown. Other orders may be used. For example, acts 62 and 64 may be performed in any sequence (e.g., act 62 first or act 64 first) or simultaneously. Similarly, acts 66 and 68/70 may be performed in any sequence or simultaneously.

Additional, different, or fewer acts may be provided. For example, acts 60-70 are performed to plan for therapy without providing the treatment in act 72. As another example, act 66 and/or acts 68 and 70 are not performed. In another embodiment, the use of a measured or surrogate motion in act 64 is not provided, such as where motion of the object is used to calculate the margin or the probability density function.

In act 60, magnetic resonance (MR) data is acquired. The MR data is acquired by scanning a patient. A sequence of pulses is transmitted into a patient subjected to a main magnetic field and any gradient fields. In response to the pulses, the spin of one or more types of atoms may vary, resulting in detectable response. The received information is reconstructed from k-space data into object or image space. In alternative embodiments, the MR data is acquired from transfer in a network or loading from memory.

Any pulse sequence or MR acquisition technique may be used. In one embodiment, the MR data is acquired with a balanced steady-state free-precession (bSSFP) MR sequence. In another embodiment, the MR data is acquired with a gradient echo MR sequence. Other 2D dynamic MRI acquisition may be used, such as half-fourier single-shot turbo spin echo (HASTE), turbo fast low angle shot (FLASH), or single-shot echo-planar imaging (EPI).

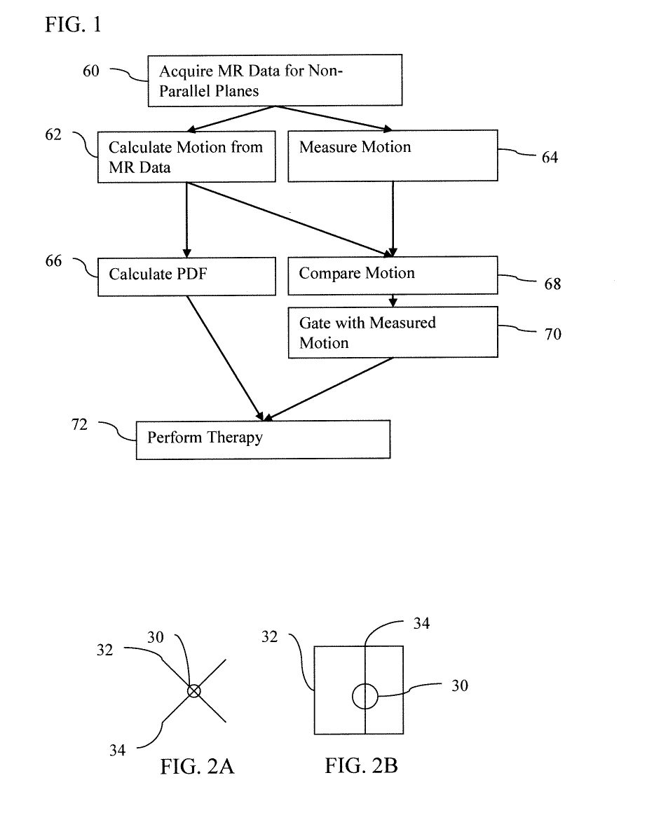

The MR data is acquired along two or more different planes. The MR data represents the response along the different planes. Raw 2D slice data in the object domain is acquired for a plurality of slice locations or planes. Each slice has a respective plane, the orientation of which may vary depending on the imaging sequence. For example, the slices may be oriented along sagittal and coronal planes, but a transversal or other orientation may be used. In one embodiment represented in FIGS. 2A and 2B, the planes 32, 34 are orthogonal. MR data is acquired along two orthogonal planes 32, 34. The planes 32, 34 are oriented so that the line or column of intersection extends generally from head-to-toe direction on the patient. “Generally” is used to account for possible offset of the patient from expected on the patient bed during scanning. The intersection may have other orientations relative to the patient. Other non-parallel relative orientation of the two planes may be used. MR data for more than two planes may be acquired, such as acquiring MR data representing three orthogonal planes.

The planes intersect the region of interest. For example, the planes are positioned to intersect an object to be subjected to therapy. The object may be a tumor, lesion, anatomical location, or other part within a patient. The intersection of the planes 32, 34 may pass through the object 30, such as represented in FIGS. 2A and 2B. The planes 32, 34 may pass through the center of the object 30, but purposeful or unintentional offset from the center of the object 30 may be used. The imaging planes 32, 34 are positioned such that at least part of the tumor or image feature to be tracked falls along their intersection.

The planes corresponding to the MR data have a thickness. The scan sequence may be associated with different possible thicknesses. The slice thickness is optimized according to out-of-plane depth of the object to be tracked to minimize errors associated with volume averaging. Since the object 30 moves due to respiratory motion and/or other causes, the thickness should be large enough to avoid losing the object 30 between sequential scans of the plane. Thicker slices may result in less contrast, so the thickness is minimized to maintain contrast. Any thickness may be used.

The acquired MR data represents the different planes at different times. The planes are each scanned a plurality of times. The scanning of each slice is repeated to acquire frames of data. Each frame of date represents the scan of the entire field of view for the slice at the desired resolution. By scanning multiple times, a plurality of frames are acquired for each slice. Multiple frames are provided for each plane location. The planes are scanned sequentially in an interleaved fashion, such as acquiring a frame for one plane, then a frame for another plane, and repeating. In other embodiments, the frames for different planes are acquired simultaneously or groups of frames are acquired for a given plane before switching to the next plane.

The MR data is acquired over multiple respiratory cycles. The number of respiratory cycles may be large, such as over tens or hundreds (e.g., 50 or 300) of cycles. For example, the image acquisition may be implemented continuously for approximately 5-30 minutes, providing data over hundreds of respiratory cycles. In another example, about 500 frames are acquired for each orientation or plane over about 4.5 minutes with an in-plane resolution for each frame of about 2×2 mm² with a 5 mm slice thickness. Shorter or longer duration and a fewer or greater number of frames may be used. As a result, the acquisition includes multiple 2D slices for each respiratory stage, segment, interval, or other portion of the respiratory cycle. The data acquisition may, but need not be, gated or otherwise timed to coincide with a respiratory cycle or phase

To increase the temporal resolution or rate of acquisition, the MR data is acquired only for the planes. Using a limited number of planes, such as two or three planes, increases the frame rate as compared to three-dimensional scanning. The MR data is provided for the planes and not other locations (e.g., no 3D scan). A pure 3D MRI acquisition may be limited by the frame rates that can be achieved. Such frame rates may not be fast enough to acquire respiratory motion in images without artifacts. Acquiring orthogonal or other non-parallel slices through the object may capture the 3D spatial motion with a significantly increased frame rate. In one example, a frame is acquired every 200-300 ms (e.g., 250 ms) by avoiding scanning the entire volume. Faster or slower frame rates may be provided. 3D scanning or scanning more than three planes may be used in other embodiments.

In act 62, the MR data is used to locate the position of the object over time. Frames of data representing the object in a plane over time may be used to determine the position in the plane or slice. By determining position in two non-parallel planes, the 3D position may be determined. The position in two directions or a 2D position is provided in each plane. Since there are two or more planes, three or more directions are provided. In one embodiment, the direction used in one plane is the same as a direction in the other plane. For example, one component of a planar coordinate system is along the intersection of the planes. The intersection is a dimension along which the position is mapped. The other direction in each plane is perpendicular to the line of intersection. Since the planes are non-parallel, the other direction in each plane is different. In orthogonal planes, the other directions are perpendicular.

The change in position over time indicates motion. The change in position along a given direction is the motion along that direction. By detecting position at different times, the motion of the object is determined. In other embodiments, the motion is determined without specifically identifying position. For example, a magnitude of motion is determined without identifying specific coordinates of the object.

The position is determined as a center of the object. A center of gravity, a geometrical center, or other center is tracked. In other embodiments, the position of different parts of the object, such as edges, is determined. Different parts of the object may move by different amounts due to compression, expansion or other distortion.

The position is determined in any of various approaches. In one embodiment, the position at each time is determined by segmenting the object or region of interest. The region of interest may include just part of the object, part of the object and part of tissue adjacent to the object, the entire object without more, or the object and surrounding tissue. Segmentation performed on each of the frames provides the position of the object at the different times.

In another embodiment, the position is determined by tracking. A reference is used to track the object in the different frames. Segmentation is used to identify the object in one of the frames as the reference. Parts of the object, the overall object, or other features may be used for tracking. Manual or automated segmentation may be used. In another embodiment, the reference is a template scaled as appropriate for the MR data. For example, MR data representing a typical tumor rather than the tumor of the patient is used as a template. The same reference is used for tracking throughout a sequence. Alternatively, the reference changes, such as using the most recently tracked frame as the reference for tracking to a next frame.

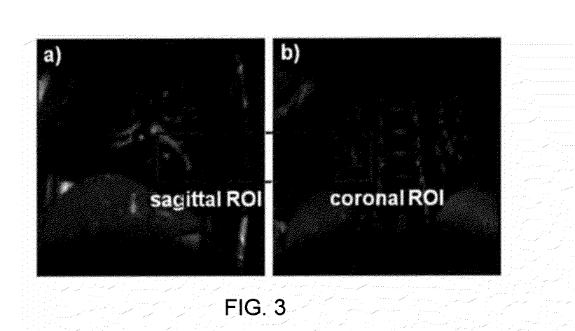

FIG. 3 shows example images from MR data used for tracking. The tracking may rely on features other than the tumor. FIG. 3 includes images from raw frames of MR data representing an abdomen. The boxes are regions of interest to be used for tracking. The vertical lines is the line of intersection. The line of intersection is positioned to pass through the lesion.

To track, the reference is correlated with each frame of data in the sequence. The reference is translated, rotated, and/or scaled to different positions relative to the frame. A correlation value is calculated at each possible position. The translation, rotation, and/or scaling with a greatest correlation indicates the position of the object. The change in position indicates motion.

Any measure of correlation may be used. For example, a normalized cross-correlation is calculated. In other examples, a minimum sum of absolute differences is calculated. Other similarity values may be used. The correlation is of the data or of features extracted from the data.

The tracking is performed for any resolution. For example, the tracking is performed at the resolution of the MR data for each frame. As another example, the frames are up-sampled, such as by interpolation. Any amount of up-sampling may be used, such as up-sampling by four times to provide 0.5 mm tracking resolution. In another example, the frames are decimated or down-sampled for tracking to reduce processing burden.

Any search pattern may be used, such as correlating for every possible position. A coarse and fine search may instead be used. The reference is correlated with relatively large steps (e.g., translate by 5-10 pixels and rotate by 10-20 degrees) between calculations. Once a greatest correlation is determined using the coarse search, relatively smaller steps may be used to refine the position. In yet another approach, knowledge about the motion is used to predict the position, and the searching is limited to a region around the predicted position. For example, position from previous cycles and the current phase within the cycle are used to predict the position or the next position.

The tracking is performed for each of the planes separately. The tracking is performed through the entire sequence of frames for each plane. In other embodiments, the tracking in one plane may be used in the tracking in another plane. For example, the position along the direction of the line of intersection from tracking in one plane is used to limit the search for tracking in another plane.

By tracking in different non-parallel planes, the position and corresponding motion is determined in three spatial dimensions. For example, motion in two directions is determined for each plane. The 2D vectors from the non-parallel planes may be combined into a 3D vector. In one embodiment, one direction in one plane is the same direction in another plane (e.g., along the intersection). Since the frames for the different planes are acquired in an interleaved manner, the position information along the intersection has a greater temporal resolution as compared to the position information along other dimensions.

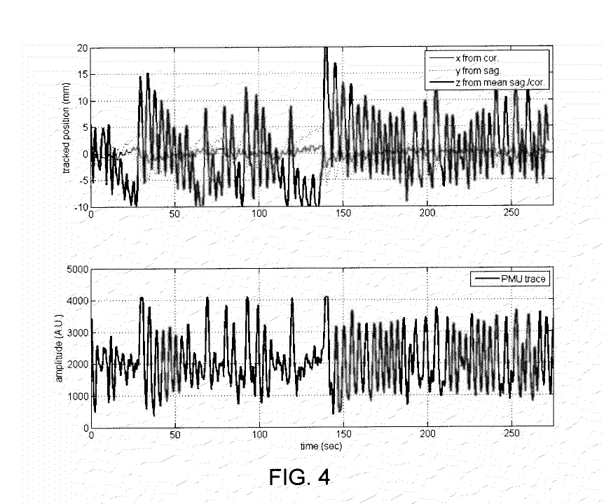

FIG. 4 shows an example position determination. The position is represented by a difference or magnitude of motion. FIG. 4 shows the position variation over time, such as 250 seconds. Position over more or less time may be determined. The three-dimensional position is determined and represented as three orthogonal components x, y, z of the 3D vector. One or two-dimensional position of the object is determined in other embodiments. The lower portion of FIG. 4 shows a synchronously acquired PMU or surrogate respiratory trace.

To increase temporal resolution, the frames used for tracking may be increased. Frames may be created by interpolation, such as interpolating to a 250 ms temporal grid. As shown in FIG. 4, the discrete position measurements may be at sufficient frequency (e.g., 250 ms) to be generally continuous. In other embodiments, the acquisition rate provides the MR data at the desired temporal grid. In another embodiment, the tracked motion is up-sampled to the desired temporal resolution. Down-sampling may be used. In FIG. 4, the z position is mapped separately for the two different frames. The z position may be mapped together as one graph with higher temporal resolution. The z measurements may be down-sampled or the x and y measurements may be up-sampled to a same temporal resolution. Alternatively, different temporal resolutions are used. In yet another embodiment, curve fitting is applied to the measurements to provide any desired temporal resolution.

The measurements of the z position may be averaged, mapped separately, or mapped together. The redundant information in the z direction may be used to check for errors. Where the z position in one plane is a threshold amount different from the z position from another plane, an error may be identified. The process may be attempted again with different settings or the user may be prompted to resolve an issue. For example, vascular features with varying signal intensity may be tracked, resulting in errors. The difference in z direction motion from the different planes may indicate this problem. The segmentation, MR data filtering, or other process may be varied to more likely track the tumor instead.

In act 64 of FIG. 1, a physiological cycle is measured. For example, the respiratory or breathing cycle is measured. Other cycles may be measured, such as the cardiac or heart cycle.

In some embodiments, the respiratory data is acquired by capturing and sampling an external respiratory surrogate signal. The measured respiratory data provides a surrogate for the respiration. The surrogate represents the breathing cycle. The cycle information may be used for gating, such as limiting treatment to particular phase or phases of the cycle. The cycle information may be used predicatively, such as assigning a likely position and/or margin as a function of the phase of the cycle. Treatment may be provided throughout the cycle, but directed to particular positions or with particular margins based on the phase of the cycle.

The measurement is independent of the position or motion determination of act 62. No MR data, the same MR data, or different MR data may be used to measure the cycle. For example, MR data indicative of respiration, or a respiratory trace, is also acquired in act 60. Respiratory data may be acquired synchronously with the image acquisition. In alternative embodiments, the variance in position over time from act 62 is used as the measurement of the physiological cycle. Any navigator imaging or self-gating technique may be used.

Respiratory data may be acquired via one or more monitors other than the MR scanner. A variety of devices or procedures may be used to generate the respiratory surrogate signal. In one example, a pneumatic belt worn by the patient is used to produce the respiratory surrogate signal. Alternative respiratory data acquisition techniques include image-based techniques, in which, for example, one-dimensional or 2D navigator (or tracking) images are acquired during the slice or volume data acquisition. The navigator images may focus on, for example, an anatomical feature in the abdomen that moves with respiration, such as the diaphragm. Other acquisition techniques include infrared (IR)-based respiratory phase monitors, such as the REAL-TIME POSITION MANAGEMENT™ (RPM) system commercially available from Varian Medical Systems, Inc., or the LED-based device on-board the CYBERKNIFE radiotherapy system used for SYNCHRONY modes of treatment delivery (Accuray, Inc.). Any one or more of these techniques may provide the surrogate data indicative of patient respiration.

The measurements of act 64 are temporally linked with the positions or frames. For example, the measurement of act 64 occurs while acquiring the frames. Time stamping or other correlation of the respiratory data and the slice data temporally relates the two. The respiratory measurement and frames of MR data may be time-stamped via a common clock.

As another example, the measured cycle and the position information are processed to define the respiratory cycles as well as a set of respiratory phase intervals, or phase bins, for each respiratory cycle. The respiratory signal and/or position may be sampled, filtered or otherwise processed to remove noise in preparation for the analysis. The sampling may include down-sampling or up-sampling. The analysis may include processing to determine the frequency of the respiratory signal (e.g., an average frequency over the imaging session) based on the sampled representation. The analysis may alternatively or additionally include the generation of a moving average representation of the respiratory signal. Some of the respiratory data may be removed from the analysis in the interest of avoiding noisy or otherwise unreliable signals. For example, the data collected during or at the end-exhale minima of the respiratory cycle may be corrupted by interference from cardiac noise and, thus, not incorporated into the analysis. In some embodiments, signal processing techniques may be alternatively or additionally used to eliminate or mitigate cardiac interference in the respiratory signal.

The analysis of the respiratory data may be used to determine a trigger or point at which the respiratory cycles may be defined as beginning. In one example, the trigger for each cycle is the peak inspiratory maximum. Other points in the respiratory cycle may alternatively be used as a trigger or cycle-defining event. Once the peak inspiratory maxima (or other trigger point) is found in each of the respiratory cycles, each respiratory cycle is segmented, discretized or otherwise divided into the set of respiratory intervals or bins. Each interval may be of equal time length for a given respiratory cycle, or may be defined on the basis of equal-likelihood over the duration of imaging. Thus, the number of intervals available in each respiratory cycle does not vary, but the width placement and ordering of the intervals may vary from cycle to cycle.

The number of respiratory intervals may be selected as a parameter of the image processing method, and may be, for example, between 8 and 15. The number may vary or be dependent, in part, on the raw imaged frame rate, or, for example, on the available 2D or 3D images. The intervals may be used for separately mapping position for the same phase, but at different cycles.

The determined position is used for therapy planning and/or application. In one embodiment represented in act 66, the position is used for planning using tumoral spatial probability density functions (PDFs). The measurement of the surrogate may not be used for PDFs. Alternatively, the measurement of the surrogate is used to determine the cycle phases associated with different positions where different probability density functions are used for different phases.

In act 66, one or more probability density functions are calculated. For example, different PDFs are provided for different phases of a respiratory cycle. As another example, gating is to be used. Accordingly, the position of the object at one phase is used. A single PDF is determined for the appropriate phase.

The PDF is used to determine dosage and spatial distribution of the dosage at different times or segments of the treatment. The probability of the tumor or other treatment region being at a given location at a phase of the cycle or time is calculated. The dosage may be controlled to more likely treat the desired object and avoid treatment of healthy tissue.

The PDF is based on the position over time. As represented in FIG. 4, the position is determined for the same phase over many cycles. For example, the tumor may be at a given 3D position 90% of the cycles, but spaced by 2 mm in a given direction 10% of the cycles. The position information is incorporated into the probability density function for the phase.

Since the position information is acquired over a long period, such as over tens or hundreds of cycles, the position information may reflect drift. For example, the respiratory drift in the position over multiple cycles is reflected by the position information. Calculating the probability of a particular position over these times can represent the drift in the position. Characterizing respiratory drift and imaging inter-fractionally allows radiation therapy treatment plans to be tailored to changes that occur over long time scales. Thus, multiple PDFs may be calculated for the therapy plan that individually take into account motion from the respiratory cycle and then, across PDFs, take into account respiratory drift. This allows tighter and more accurate treatment volumes to better irradiate tumors and spare healthy tissue.

In an additional or alternative use of the planar tracking of the object, the appropriateness of gating for a given patient is determined. In act 68, the motion of the object is compared to the surrogate motion (i.e., motion measured in act 64). The comparison is used to determine whether the position variation of the object in the cycle results in inaccuracies in the gated treatment. Some patients may have sufficient variation that the treatment is less likely to be applied to the desired object, so other approaches than gating based on measures of surrogate motion should be used.

The amount of offset of the position based on the tracking (act 62) from the position based on the measurement (act 64) is determined. The offset in position may be expressed as motion. The offset in position may be an offset in amount of motion. The motion may be compared using one or more of different approaches. The magnitude of motion, motion vector, magnitude of change of position, the vector for change in position, the position, or variance may be compared.

The comparison may be along a specific direction (1D), within a plane (2D), or for a volume (3D). For example, the motion along the intersection (e.g., z or head-to-toe direction) is used for comparison without using motion in other directions. Acquisition of consecutive orthogonal 2D slices allows for substantially continuous tracking of one direction of motion and forms a surrogate of tumor motion which may be used for comparison with external surrogate captured motion.

The comparison is a difference. The difference in magnitude, vector difference, or distance apart is calculated. Any difference function may be used alone or with other variables. The difference is determined for each time. The differences over time may be averaged. Any combination of the differences may be calculated. Alternatively, each difference is separately compared to avoid any one time where the surrogate motion would be overly inaccurate.

Using vector differences, one difference for each time is determined. In other embodiments, the differences for each direction may be combined. The differences from different directions are kept separate or combined together for each time or as an overall measurement. Any combined differences for different directions may be combined across directions or used separately.

Simultaneous capture of external surrogates, such as a respiratory belt, with object tracking allows for the direct comparison of tumor motion with that detected by external surrogates. The comparison provides assessment of whether gating treatment methods are feasible in a specific patient.

In act 70, gating-based treatment is performed when the motion of the object is within a threshold of the surrogate motion. Multiple differences are thresholded by a same threshold. Alternatively, different thresholds are applied for different times or different combinations of differences. The thresholding may be part of fuzzy logic or other filtering for determining whether gating techniques relying on the surrogate measure of motion are appropriate. The results of the comparison or comparisons indicate whether gating-based treatment should be used or not and/or indicate additional risk or not associated with the use of gating-based treatment.

Where the amount of offset between the object motion and the surrogate motion is below the threshold, gating may be allowed. By comparing over a long period any drift may result in a larger difference or offset. Where such a larger difference does not occur, there may be little drift. In these patients with consistent motion, the treatment is more likely to be directed to the desired object.

Where the amount of offset between the object motion and the surrogate motion is above the threshold for a given time or over all or some times, gating may not be allowed. Drift or other causes may indicate that the surrogate motion is not accurate to some level. The lack of accuracy may result in the risk of healthy tissue being damaged and/or the object to be treated receiving less than the desired dose. Depending on the level of risk and the patient's medical situation, the treatment may not be allowed.

The decision may be of allowing treatment or not. This decision is made in light of the risk due to drift or other inaccuracy in surrogate motion as compared to object motion. The system or program may disable or enable treatment based on the differences. Alternatively, the differences or a level of risk is output to the user for decision making. An indication of offset, comparison of the offset to the threshold, range or size of the offset, timing of the offset, or other information associated with differences between the surrogate motion and the object motion may be output. The output is used by the physician or others to allow or not allow gating-based treatment.

Other therapy planning may benefit from the tracked motion of the object. For example, the margin size may adapt to the variance in motion for a given phase, over a cycle, or over multiple cycles.

FIG. 5 shows a system 10 for therapy planning using image tracking. The system 10 includes a cryomagnet 12, gradient coils 14, whole body coil 18, local coil 16, patient bed 20, MR receiver 22, processor 26, memory 28, monitor 29, and therapy device 24. Additional, different, or fewer components may be provided. For example, another local coil or surface coil is provided for signal reception other than the local coil 16. As another example, servers or other processors may be provided for data processing.

Other parts of the MR system are provided within a same housing, within a same room (e.g., within the radio frequency (RF) cabin), within a same facility, or connected remotely. The other parts of the MR portion may include local coils, a cooling system, a pulse generation system, an image processing system, a display, and a user interface system. Any now known or later developed MR imaging system may be used with the modifications discussed herein, such as a 1.5T Siemens System (MAGNETOM Espree).

The location of the different components of the MR system is within or outside the RF cabin, such as the image processing, tomography, power generation, and user interface components being outside the RF cabin. Power cables, cooling lines, and communication cables connect the pulse generation, magnet control, and detection systems within the RF cabin with the components outside the RF cabin through a filter plate.

The MRI system is a scanner. The scanner is configured to scan along different planes, such as orthogonal planes, for object tracking. The scan avoids other locations for object tracking to increase the repetition frequency of the scanning. The scanner acquires frame data over the plurality of respiratory cycles. By interleaving, the frame data includes a plurality of frames for each of the different planes.

For the MRI scanner, the cryomagnet 12, gradient coils 14, and body coil 18 are in the RF cabin, such as a room isolated by a Faraday cage. A tubular or laterally open examination subject bore encloses a field of view. A more open arrangement may be provided. The patient bed 20 (e.g., a patient gurney or table) supports an examination subject such as, for example, a patient with a local coil arrangement, including the coil 16. The patient bed 20 may be moved into the examination subject bore in order to generate images of the patient. Received signals may be transmitted by the local coil arrangement to the MR receiver 22 via, for example, coaxial cable or radio link (e.g., via antennas) for localization.

In order to examine the patient, different magnetic fields are temporally and spatially coordinated with one another for application to the patient. The cyromagnet 12 generates a strong static main magnetic field B₀ in the range of, for example, 0.2 Tesla to 3 Tesla or more. A resistive or other magnet may be used. The main magnetic field B₀ is approximately homogeneous in the field of view.

The nuclear spins of atomic nuclei of the patient are excited via magnetic radio-frequency excitation pulses that are transmitted via a radio-frequency antenna, shown in FIG. 5 in simplified form as a whole body coil 18, and/or possibly a local coil arrangement. Radio-frequency excitation pulses are generated, for example, by a pulse generation unit controlled by a pulse sequence control unit. After being amplified using a radio-frequency amplifier, the radio-frequency excitation pulses are routed to the body coil 18 and/or local coils 16. The body coil 18 is a single-part or includes multiple coils. The signals are at a given frequency band. For example, the MR frequency for a 3 Tesla system is about 123 MHz +/−500 KHz. Different center frequencies and/or bandwidths may be used.

The gradient coils 14 radiate magnetic gradient fields in the course of a measurement in order to produce selective layer excitation and for spatial encoding of the measurement signal. The gradient coils 14 are controlled by a gradient coil control unit that, like the pulse generation unit, is connected to the pulse sequence control unit. The gradient coils 14 are used to control scanning of just the desired planes, such as orthogonal planes.

The signals emitted by the excited nuclear spins are received by the local coil 16. In some MR tomography procedures, images having a high signal-to-noise ratio (SNR) may be recorded using local coil arrangements (e.g., loops, local coils). The local coil arrangements (e.g., antenna systems) are disposed in the immediate vicinity of the examination subject on (anterior) or under (posterior) or in the patient. The received signals are amplified by associated radio-frequency preamplifiers, transmitted in analog or digitized form, and processed further and digitized by the MR receiver 22.

The MR receiver 22 connects with the coil 16. The connection is wired (e.g., coaxial cable) or wireless. The connection is for data from the coil 16 to be transmitted to and received by the MR receiver 22. The data is K-space data. In response to an MR pulse, the coil 16 generates the K-space data and transmits the data to the MR receiver 22. Any pulse sequence may be used, such as a pulse sequence acquiring projections along two or three spatial axes. Any spatial resolution may be provided, such as a spatial resolution of 0.78 mm.

The MR receiver 22 includes the processor 26 or another processor (e.g., digital signal processor, field programmable gate array, or application specific circuit for applying an inverse Fourier transform) for reconstructing object space data from K-space data. The MR receiver 22 is configured by hardware or software to calculate X, Y, and Z MR data from the K-space data from the coil 16. The recorded measured data is stored in digitized form as complex numeric values in a k-space matrix. An associated MR image of the examination subject may be reconstructed using a one or multidimensional Fourier transform from the k-space matrix populated with values. For position tracking, the reconstructed MR data may be used without or in addition to generating an image. Other transforms for reconstructing spatial data from the K-space data may be used.

The monitor 29 is a respiratory monitor. The monitor 29 acquires surrogate respiratory data over a plurality of respiratory cycles. Measurements of the motion or position of tissue (e.g., skin or chest) are performed over time. The measured location responds to the diaphragm or lungs, so represents the respiratory cycle.

In one embodiment, the monitor 29 is the MRI scanner. Using navigation images or self-gating techniques, the motion of the lungs is determined. This determination is separate from imaging, but may use MR data or k-space data also used for tracking. The monitor 29 measures as the MR scanner acquires frames of data for the planes.

In another embodiment, the monitor 29 is a different sensor than the MR scanner. For example, a camera is used to detect chest motion. As another example, a respiratory belt is used. In another example, an exhalation sensor (e.g., infra-red or temperature sensor) is used.

The respiratory monitor 29 is configured to acquire respiratory surrogate data over a plurality of respiratory cycles. One or more of the processors 26 is in communication with the respiratory monitor 29 and the receiver 22 to implement the methods described above.

The processor 26 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining position. The processor 26 is a single device or multiple devices operating in serial, parallel, or separately.

The processor 26 is in communication with the respiratory monitor 29 and the receiver 22 of the MR scanner. The processor 26 and memory 28 may be part of a medical imaging system, such as the MR system. In one embodiment, the processor 26 and memory 28 are part of the MR receiver 22. Alternatively, the processor 26 and memory 28 are part of an archival and/or image processing system, such as associated with a medical records database workstation or server. In other embodiments, the processor 26 and memory 28 are a personal computer, such as desktop or laptop, a workstation, a server, a network, or combinations thereof. The processor 26 and memory 28 may be provided without other components for implementing the method.

As part of the MR receiver 22, the processor 26 applies an inverse Fast Fourier transform to calculate the power spectrum of the k-space data. The power spectrum provides intensity as a function of frequency. The frequency corresponds to space or distance. The MR data as acquired is a function of frequency and after applying inverse FT becomes a function of space.

The processor 26 is configured by instructions, design, hardware, and/or software to perform the acts discussed herein. The processor 26 is configured to determine motion in the different planes. The motion is determined based on position tracking for each plane. The frames of data for each plane are used to track an object position over time. The position may be a relative position (e.g., moved 2 mm at 20 degrees) or an absolute position (e.g., at x, y, z). Since the frames represent the plane at different times, the position over time is determined. The determination is along one, two, or three axes. In one embodiment, the motion in both planes is tracked along a common direction, such as motion along an intersection of the planes. The motion is of the object at the intersection or of the object in the plane and along the direction of the intersection.

The processor 26 is configured to calculate differences between the motion determined from the frames and a motion from the surrogate respiratory data over multiple respiratory cycles. The difference is of position, motion, or cycle. The difference may be of one cycle, such as a cycle likely associated with drift. The difference may be based on multiple cycles, such as an average difference. The difference may be based on multiple measures in a same cycle, such as an average over the cycle. Any combination of differences may be used. Any difference function may be used, such as phase shift or a difference of integrals.

The processor 26 is configured to indicate a feasibility of gating and/or tracking treatment based on the differences. The indication is a displayed output. The output is of the differences, the relationship of the difference to a threshold, enabling of treatment, or disabling treatment. The indication may be a signal, such as an enable or disable signal for controlling the therapy device 24. In one embodiment, the indication is output as feasible when the differences indicate a drift (e.g., average difference) over the respiratory cycles below a threshold and as infeasible when the differences indicate the drift above the threshold.

The processor 26 is configured to calculate a probability density function as a function of the determined motion. Using the position over time, the location of the object at different times is used to determine the likelihood that the object is at each location. The center of the object may be used. In other embodiments, the edges of the object are identified and used. Any probability density function calculation may be used.

The memory 28 is a graphics processing memory, a video random access memory, a random access memory, system memory, random access memory, cache memory, hard drive, optical media, magnetic media, flash drive, buffer, database, combinations thereof, or other now known or later developed memory device for storing MR data or image information. The memory 28 is part of an imaging system, part of a computer associated with the processor 26, part of a database, part of another system, a picture archival memory, or a standalone device.

The memory 28 stores K-space data, reconstructed MR data, templates, measured surrogate information, and/or object position or motion information. The memory 12 or other memory is alternatively or additionally a computer readable storage medium storing data representing instructions executable by the programmed processor 26 for therapy planning using magnetic resonance imaging (MRI). The instructions for implementing the processes, methods and/or techniques discussed herein are provided on non-transitory computer-readable storage media or memories, such as a cache, buffer, RAM, removable media, hard drive or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

A display may be provided for indicating the position, position over time, indication of risk, probability density function, allowance of gating-base treatment, MR images, or other information. The display is a monitor, LCD, projector, plasma display, CRT, printer, or other now known or later developed devise for outputting visual information. The display receives images, graphics, or other information from the processor 26 or memory 28.

The therapy device 24 is a medical device for applying radiation, particles, ultrasound, heat, current, or other energy for treatment. For example, the therapy device 24 is an x-ray source for radiating a tumor. As another example, the therapy device 24 is an ultrasound transducer for generating heat with focused acoustical energy at the object. The therapy device 24, using focus, aperture, collimation, or other technique, directs energy to the treatment location and not other locations.

The therapy device 24 is mounted to the MRI system. For example, the x-ray source is provided on a gantry connected around the patient aperture of the MRI system. As another example, an ultrasound transducer is provided in the patient bed 20. In alternative embodiments, the therapy device 24 is separate from the MRI system, such as being a hand held, patient worn, or robotically controlled therapy device 24.

The therapy device 24 is in communication with the processor 26. The communication with the processor 26 may also be used to enable or not the gated therapy. The dosage, dose sequence, and/or therapy plan are provided to the therapy device 24 for implementation. The therapy plan is created as known or later developed, but may be based on a probability density function using the tracked location of the object. The therapy plan may use gating, increased margin, or other approach based on the object motion. Based on communication from the monitor 29, the operation of the therapy device 24 may be controlled to gate the therapy.

While the invention has been described above by reference to various embodiments, it should be understood that many changes and modifications may be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for therapy planning using magnetic resonance imaging (MRI), the method comprising:
acquiring magnetic resonance (MR) data representing first and second planes at different times over a plurality of breathing cycles, the first and second planes intersecting through an object in a patient and being non-parallel;
tracking, with a processor, a position of the object along first and second directions in the first plane from the MR data representing the first plane;
tracking, with a processor, the position of the object along third and fourth directions in the second plane from the MR data representing the second plane;
measuring the breathing cycle;
comparing, with the processor, a position based on the measured breathing cycle with the position as tracked over time in at least the second direction;
determining an allowance or not of therapy using the measuring of the breathing cycle based on the comparing; and
incorporating the position from the tracking into a probability density function for the therapy.

2. The method of claim 1 wherein acquiring comprises acquiring the MR data just for the first and second planes and not other locations such that MR data is provided for each of the first and second planes every at least 300 milliseconds.

3. The method of claim 1 wherein acquiring comprises acquiring the MR data with a balanced steady-state free-precession MR sequence, a gradient echo MR sequence, or a spin echo MR sequence.

4. The method of claim 1 wherein tracking in the first and second planes comprises tracking with the second and third directions being a same direction along a line of intersection of the first plane with the second plane.

5. The method of claim 4 wherein tracking along the line of intersection comprises tracking along a head-to-toe axis of the patient, the line of intersection oriented to be along the head-to-toe axis.

6. The method of claim 1 wherein tracking in the first and second planes each comprises two-dimensional tracking with correlation of the MR data from different times.

7. The method of claim 1 wherein measuring the breathing cycle comprises measuring a breathing cycle with a respiratory belt, navigator images or self-gating.

8. The method of claim 1 wherein comparing comprises determining an amount of offset of the position based on the tracking from the position based on the measuring, and wherein determining comprises allowing where the amount of offset is below a threshold.

9. The method of claim 4 wherein comparing comprises comparing the position along the line of intersection with the position based on the measuring.

10. The method of claim 1 wherein incorporating comprises accounting for respiratory drift in the position in the probability density function.

11. The method of claim 1 wherein incorporating comprises incorporating different locations for the position at a same phase of different cycles.

12. A system for therapy planning using image tracking, the system comprising:
a respiratory monitor to acquire surrogate respiratory data over a plurality of respiratory cycles;
a scanner to acquire frame data over the plurality of the respiratory cycles, the frame data comprising first and second pluralities of frames representing first and second orthogonal planes, respectively, at different times; and
one or more processors in communication with the respiratory monitor and the scanner, the one or more processors being configured to determine motion in the first and second planes from the first and second pluralities of the frames, respectively, to calculate differences between the motion determined from the frames and a motion from the surrogate respiratory data over the plurality of the respiratory cycles, and to indicate a feasibility of gating treatment or motion tracking based on the differences.

13. The system of claim 12 wherein the respiratory monitor comprises a respiratory belt.

14. The system of claim 12 wherein the scanner comprises a magnetic resonance scanner configured to scan along the first and second planes and not elsewhere for acquiring the frames used for determining the motion.

15. The system of claim 12 wherein the one or more processors are configured to determine the motion in the first and second planes in a same direction along an intersection of the first plane with the second plane.

16. The system of claim 12 wherein the one or more processors are configured to indicate the feasibility as feasible when the differences indicate a drift over the respiratory cycles below a threshold and indicate the feasibility as infeasible when the differences indicate the drift above the threshold.

17. The system of claim 12 wherein the one or more processors are configured to calculate a probability density function as a function of the determined motion.

18. In a non-transitory computer readable storage medium having stored therein data representing instructions executable by a programmed processor for therapy planning using magnetic resonance imaging (MRI), the storage medium comprising instructions for:
locating position as a function of time of an object represented in MR data for different planes through a patient;
calculating spatial probability density functions for different phases of a respiratory cycle as a function of the position over the time; and
accounting for drift over respiratory cycles including the respiratory cycle as a function of the position over the time.

19. The non-transitory computer readable storage medium of claim 18 wherein locating the position comprises tracking the object with correlation through a first sequence of frames representing a first of the different planes and tracking the object with correlation through a second sequence of frames representing a second of the different planes, such that the position is determined in three spatial dimensions.

20. The non-transitory computer readable storage medium of claim 18 wherein locating comprises calculating motion of the object;
further comprising:
comparing the motion of the object to a surrogate motion; and
gating treatment when the motion of the object is within a threshold of the surrogate motion.
